(19) United States
(12) Patent Application Publication
(10) Pub. No.: US 2022/0287830 A1
(43) Pub. Date: Sep. 15, 2022

(54) Expandable Stent For A Transcatheter Prosthetic Heart Valve

(71) Applicant: St. Jude Medical, Cardiology Division, Inc., St. Paul, MN (US)

(72) Inventor: William H. Peckels, Robbinsdale, MN (US)

(73) Assignee: St. Jude Medical, Cardiology Division, Inc., St. Paul, MN (US)

(21) Appl. No.: 17/688,152

(22) Filed: Mar. 7, 2022

Related U.S. Application Data
(60) Provisional application No. 63/158,576, filed on Mar. 9, 2021.

(51) Int. Cl. A61F 2/24

(52) U.S. Cl. CPC A61F 2/2418; A61F 2/2415; A61F 2230/0008; A61F 2220/0058; A61F 2220/0083

(57) ABSTRACT

A stent includes a body portion having a first configuration and a second configuration. The body portion in the first configuration lies substantially in a plane and may be curved about an axis substantially perpendicular to the plane. The planar body portion may be manipulated to the second configuration that is nonplanar.

### CROSS-REFERENCE TO RELATED APPLICATIONS

The present application claims the benefit of the filing date of U.S. Provisional Patent Application No. 63/158,576 filed Mar. 9, 2021, the disclosure of which is hereby incorporated by reference herein.

### FIELD OF THE INVENTION

The present disclosure relates generally to the field of stents, and in particular, to stents for use in transcatheter prosthetic heart valves. More particularly, the present disclosure is directed to improved methods for manufacturing such stents.

### BACKGROUND

Prosthetic heart valves, including surgical heart valves and expandable heart valves intended for transcatheter aortic valve replacement (“TAVR”) or transcatheter mitral valve replacement (“TMVR”), are well known in the patent literature. (See U.S. Pat. Nos. 3,657,744; 4,056,854; 5,411,552; 5,545,214; 5,855,601; 5,957,948; 6,458,153; 6,540,782; 7,510,575; 7,585,321; 7,682,390; and 9,326,856; and U.S. Pub. No. 2015/0320556.) Surgical or mechanical heart valves may be sutured into a native heart valve annulus of a patient during an open-heart surgical procedure, for example. Expandable heart valves may be delivered into a patient via a tube-like delivery apparatus such as a catheter, a trocar, a laparoscopic instrument, or the like to avoid a more invasive procedure such as full open chest, open heart surgery. As used herein, reference to an “expandable” heart valve includes those that are self-expending and those that are mechanically expandable via, for example, a balloon. Often the term “collapsible/expandable” heart valve is used herein and unless the text or the context dictate otherwise, this term is meant to include heart valves that may be collapsed to a small cross-section that enables them to be delivered into a patient through a tube-like delivery apparatus in a minimally invasive procedure, and then self-expanded or mechanically expanded to an operable size once in place.

Prosthetic heart valves typically take the form of a one way valve structure (often referred to herein as a valve assembly) mounted to/within a stent. In general, expandable heart valves include a self-expanding or balloon expandable stent, often made of a metal, such as Nitinol or stainless steel. The one way valve assembly mounted to/within the stent includes one or more leaflets, and may also include a cuff or skirt. The cuff may be disposed on the stent's interior or luminal surface, its exterior or abluminal surface, and/or on both surfaces. (See U.S. Pat. Nos. 6,458,153; 7,585,321; 8,992,608; 9,241,794; and 9,289,296; and U.S. Pub. No. 2015/0320556.) The cuff ensures that blood does not just flow around the valve leaflets if the valve or valve assembly are not optimally seated in a valve annulus. A cuff, or a portion of a cuff, disposed on the exterior of the stent can help retard leakage around the outside of the valve (the latter known as paravalvular or “PV” leakage).

Leaflets, cuffs and valve assemblies for prosthetic heart valves may be derived from various natural tissues or synthetic materials. Commercial natural tissues that have been chemically treated or “fixed” are often used. For example, leaflets could be made of bovine pericardium and cuffs could be made of porcine pericardium. (See, e.g., U.S. Pat. No. 5,957,949 at 6:23 33; U.S. Pat. No. 6,458,153 at 8:28 40; U.S. Pat. No. 5,855,601 at 6:21 30; and U.S. Pat. No. 7,585,321 at 13:5 36.) Other materials that may be used to form valve components include various synthetic polymers including, without limitation, polytetrafluoroethylene (PTFE) or polyester (see U.S. Pat. No. 5,855,601 at 6:29-31; U.S. Pat. Nos. 10,039,640; 10,022,211; 9,056,006; and 10,299,915; and U.S. Pub. Nos. 2018/0055632; 2017/0258585; 2018/0078368; and 2019/0201190), and elastic materials including silicone rubber and polyurethanes. (See U.S. Pat. No. 6,540,782 at 6:2-5.) These materials have been used in the form of continuous sheets, porous felts (U.S. Pat. No. 6,540,782 at 6:17-23) or woven fabrics. (See also U.S. Pat. Nos. 10,039,640; 10,299,915; 10,022,211; and 4,610,688; and U.S. Pub. Nos. 2018/0055632; 2017/0258585; and 2018/0078368; see also Basir et al., “Flexible mechanoprosthesis made from woven ultra-high-molecular-weight polyethylene fibers; proof of concept in a chronic sheep model”; Interactive CardioVascular and Thoracic Surgery, 25(2017) 942-949; and Yamagishi and Kurosawa, “Outflow Reconstruction of Tetralogy of Fallot Using a Gore-Tex Valve”; Ann. Thorac Surg. 1993; 56:1414-17.) Valve components and valve assemblies may be attached to a collapsible/expandable stent or frame by sutures or may be molded, glued, or soldered to the stent. (See U.S. Pat. No. 7,585,321 at 13:30-31.)

Current methods of manufacturing expandable stents used in prosthetic heart valves involve laser cutting a desired shape from a round tube of metal. The cut tube can then be expanded to a desired shape, and the expanded shape can then be shape set by, for example, being placed within a molten salt bath.

This manufacturing process requires a large amount of precision and care given the size and delicate structure of the stent. Even minor deviations from the desired expanded shape of the stent can have disastrous consequences for a patient after implantation. As such, any inaccuracies during manufacturing can have an outsized effect on the structure of the stent and its efficacy within a patient.

### BRIEF SUMMARY

One aspect of the disclosure provides a stent comprising a body portion having a first configuration and a second configuration, the body portion in the first configuration lying substantially in a plane and having a first end, a second end, a first attachment point at the first end, and a second attachment point at the second end, the body portion in the first configuration being curved about an axis substantially perpendicular to the plane.

Another aspect of the disclosure provides a method of forming a stent, the method comprising cutting a body portion of the stent from a substantially planar plate, the body portion in a first configuration being substantially planar and having a first end, a second end, a first attachment point at the first end, and a second attachment point at the second end, and connecting the first attachment point to the second attachment point to transform the body portion from the first configuration to a second configuration that is nonplanar.

### BRIEF DESCRIPTION OF THE DRAWINGS

FIG. 1 is an illustration of a stent in an initial flat, expanded configuration in accordance with aspects of the disclosure.

FIG. 2 is a top perspective view of the stent of FIG. 1 in a final three-dimensional, expanded configuration.

FIG. 3 is a side view of the stent of FIG. 1 in a final three-dimensional, compressed configuration.

FIG. 4 is an illustration of a joined portion of the stent of FIG. 1 in accordance with aspects of the disclosure.

FIG. 5 is an illustration of a joined portion of the stent of FIG. 1 in accordance with aspects of the disclosure.

FIG. 6 is an illustration of a joined portion of the stent of FIG. 1 in accordance with aspects of the disclosure.

FIG. 7 is an illustration of another embodiment of a stent in an initial flat, compressed configuration in accordance with aspects of the disclosure.

FIG. 8 is an illustration of another embodiment of a stent in an initial flat, expanded configuration, in accordance with aspects of the disclosure.

### DETAILED DESCRIPTION

Cutting an expandable stent from a round tube requires extremely precise control of the linear and rotational movement of the tube, as well as the cutter, during the cutting process. As such, cutting a stent from a round tube requires more moving parts and is more complex than cutting the stent from a flat plate. Such increases in complexity increase inefficiencies and decrease accuracy during manufacture of the stent. This disclosure is directed to methods of manufacturing a stent that minimize such difficulties.

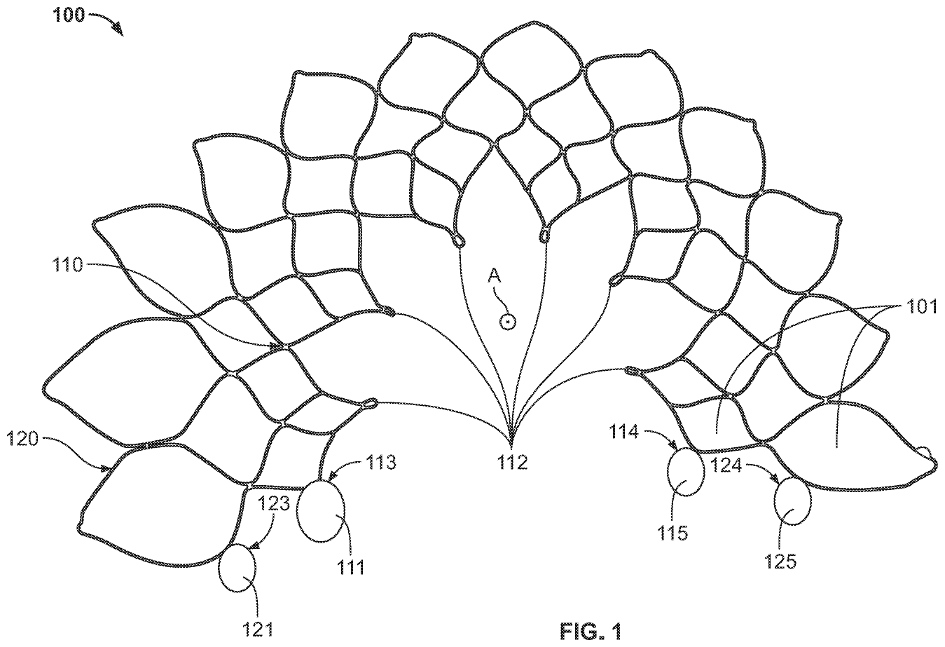

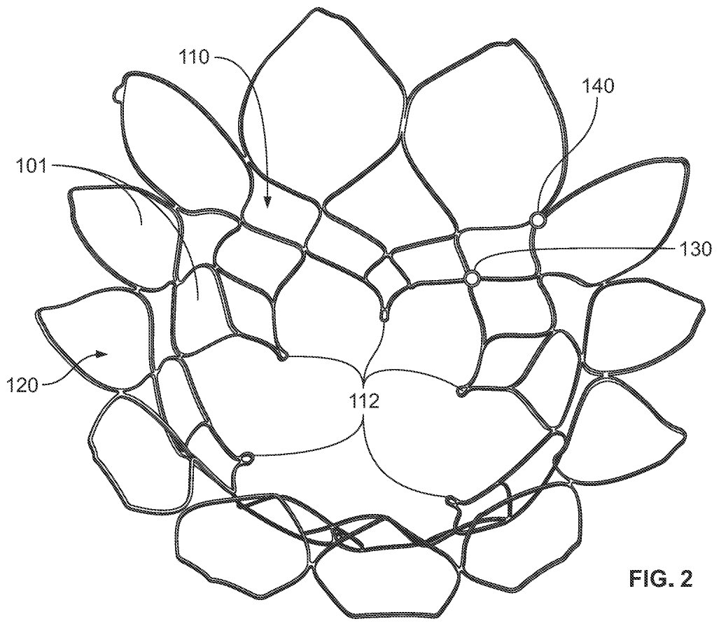

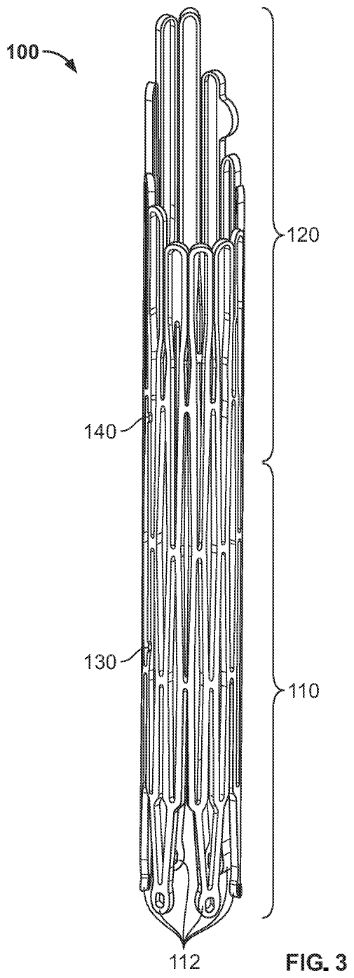

FIGS. 1-3 depict a stent 100 having a body portion 110 and a cuff portion 120. FIG. 1 depicts stent 100 in an initial configuration having a substantially planar expanded profile. FIGS. 2-3 depict stent 100 in a final or finished configuration in which the stent has a non-planar profile in both an expanded condition and a compressed condition, respectively.

Stent 100 may be formed from one or more of a variety of suitable biocompatible materials. For example, in some embodiments, stent 100 may be made of polyethylene, or other hard or semi-hard polymer, and may be covered with a polyester velour to promote ingrowth. In other examples, stent 100 may be made of metal, such as, for example, Nitinol, stainless steel, titanium and other biocompatible metals.

Stent 100 includes a number of cells each having an opening 101 therein. This enables stent 100 to be flexible such that the stent in the final configuration can be collapsed from an expanded condition, as depicted in FIG. 2, to a compressed condition, as depicted in FIG. 3. In the compressed condition, stent 100 may be, for example, inserted within a catheter for delivery into and implantation within a patient. Stent 100 may then be expanded again once the stent has been deployed within the patient (e.g., through the heat of the patient's body triggering the shape set properties of the stent).

Body portion 110 may also have a number of cells each having an opening 101 therein that is smaller than those of cuff portion 120. In such embodiments, cuff portion 120 may encompass a larger surface area than body portion 110. This may be particularly helpful as cuff portion 120 can be in more direct contact with a patient's anatomy, such as a native valve annulus of the patient's heart, to ensure that stent 100 is secured within the heart. In other embodiments, the openings 101 of cuff portion 120 may be equal or smaller in size than the openings of body portion 110.

Body portion 110 includes engagement portions 112 that may extend from an end of the body portion opposite cuff portion 120 and within the planar profile of stent 100. In some embodiments, each engagement portion 112 may have an aperture therein. This aperture can allow an additional component (not shown) to be assembled to stent 100. For example, a suture, tether, inner frame, or covering can be assembled to engagement portions 112. In other embodiments, the engagement portions may not include an aperture. Engagement portions 112 may remain at the same radial distance from a central longitudinal axis of stent 100 when the stent is in the final non-planar configuration. In other embodiments, engagement portions 112 may extend from body portion 110 in any direction, such as in radial directions relative to the central longitudinal axis of the stent when in the final non-planar configuration.

FIG. 1 depicts stent 100 in an initial expanded configuration, for example, immediately after being cut from a substantially planar plate (not shown), or after being cut and expanded. Stent 100 may be cut from the plate using any cutting method known in the art, such as laser cutting, mechanical cutting, computer numerical control (CNC), or the like. As stent 100 is cut from a substantially planar plate, the stent initially has a substantially planar profile. In the embodiment shown in FIG. 1, stent 100 has a profile that is curved within a plane. That is, stent 100 is curved about an axis A that is substantially perpendicular to the plane. However, that need not be the case, as other planar profiles are also contemplated, as discussed below.

Body portion 110 may be curved about axis A so as to have a curved length defined between ends 113 and 114 of the body portion. Cuff portion 120 may be curved about axis A so as to have a curved length defined between ends 123 and 124 of the cuff portion. The curved length in cuff portion 120 is greater than the curved length in body portion 110 so that, when manipulated into the final expanded configuration, stent 100 has a frustoconical profile, as discussed further below.

Body portion 110 includes a first attachment point 111 at end 113 and a second attachment point 115 at end 114, while cuff portion 120 includes a third attachment point 121 at end 123 and a fourth attachment point 125 at end 124. However, stent 100 may include any number of pairs of attachment points, such as one, two, three, four or more pairs of attachment points.

Attachment points 111, 115, 121, 125 are depicted simply as abstract representations in FIG. 1. However, the attachment points have structure that enables them to be secured together. For example, first attachment point 111 may be configured to engage with second attachment point 115 to secure ends 113 and 114 of body portion 110 together. Similarly, third attachment point 121 may be configured to engage with fourth attachment point 125 to secure ends 123 and 124 of cuff portion 120 together. In this manner, stent 100 may be cut from a substantially planar plate to have an initially flat configuration, and may then be manipulated so that attachment points 111, 121 engage with attachment points 115, 125, respectively, to form joined portions 130, 140 in the final configuration of the stent, as shown in FIGS. 2-3. In some embodiments, there may be more than one pair of attachment points on each of body portion 110 and cuff portion 120, such as two, three, or more pairs of attachment points on each portion. Alternatively, either body portion 110 or cuff portion 120 may have no attachment points. Further, the number of attachment points on body portion 110 may be different from the number on cuff portion 120.

FIG. 2 depicts stent 100 in a final expanded configuration. This configuration may be formed by joining respective attachment points 111, 115 and 121, 125 of stent 100 in the initial expanded configuration, depicted in FIG. 1, to form joined portions 130, 140. Specifically, first attachment point 111 may be secured to third attachment point 115 to form joined portion 130, and second attachment point 121 may be secured to fourth attachment point 125 to form joined portion 140.

FIG. 3 depicts stent 100 in a final compressed configuration in which the openings 101 in stent 100 are smaller than in the final expanded configuration. In other words, stent 100 in the expanded condition depicted in FIG. 2 can be collapsed to the compressed condition depicted in FIG. 3. In this compressed condition, stent 100 may be received within a catheter to be delivered into, and implanted within, a patient. Once stent 100 reaches the desired location within the patient's anatomy, the stent may be expelled from the catheter. No longer constrained by the catheter, stent 100 may automatically convert to the expanded frustoconical configuration shown in FIG. 2.

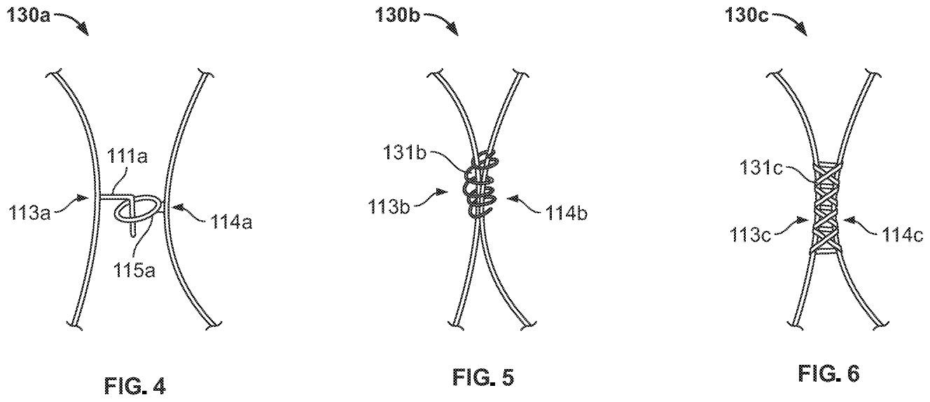

FIGS. 4-6 depict examples of joined portion 130, illustrating the engagement between ends 113 and 114 of body portion 110. It should be understood that joined portion 140 may have a similar structure as joined portion 130, however, only joined portion 130 will be discussed for the sake of brevity.

FIG. 4 depicts joined portion 130a with end 113a having a first attachment point in the form of a hook 111a extending therefrom and end 114a having a second attachment point in the form of a receptacle 115a extending therefrom. Receptacle 115a has an aperture sized to receive hook 111a. By inserting hook 111a into receptacle 1154a, end 113a can be secured to end 114a. In other examples, hook 111a may be on end 114a and receptacle 115a may be on end 113a.

FIG. 5 depicts joined portion 130b with ends 113b and 114b secured together with a knot 131b. Knot 131b may be any knot known in the art, and may be made of suture, string, or the like.

FIG. 6 depicts joined portion 130c with end 113c integrally joined to end 114c. Such an engagement may be achieved by welding, soldering, or brazing ends 113c, 114c to each other, or by another known technique. The welding, soldering, and brazing processes may use any filler material known in the art to achieve an integral attachment.

A method of manufacturing stent 100 will now be described. A substantially planar plate will first be received within a cutting machine (e.g., a laser cutting machine or the like). The cutting machine may cut the plate to form stent 100 in an initial configuration. Stent 100 in the initial configuration may be in either an expanded condition, or it may be in a compressed condition and subsequently expanded. Stent 100 may be cut from the plate so as to include attachment points 111, 115, 121, 125, or, in other methods, the stent may be cut from the plate without the attachment points, and the attachment points may later be secured to the stent, such as, for example, by suturing, welding or another attachment method. As noted, when stent 100 is cut from the plate in the compressed condition, it may subsequently be expanded. While maintaining a substantially planar profile, stent 100 may be expanded by pulling end 113 away from end 114, and end 123 away from end 124. Expansion may also be accomplished through the use of dies or other processes known in the art to change the shape of a stent. While being expanded, and while maintaining the substantially planar profile, stent 100 may also be curved about an axis perpendicular to the plane (i.e., axis A) to achieve the expanded shape shown in FIG. 1.

While stent 100 is in the initial expanded configuration, additional components may be assembled to the stent. In this planar configuration, it is far easier to assemble certain components to stent 100 than it is when the stent is in a non-planar configuration, such as when the stent is in a frustoconical configuration. For example, a skirt or cuff (either a “covering”) (not shown) may be assembled to the interior surface, the exterior surface or both surfaces of stent 100. The covering may be formed from one or more synthetic materials, such as polyester, from organic tissue, or from a combination of synthetic materials and tissue to provide adequate sealing of the stent and to promote tissue ingrowth once the stent has been implanted. For example, the tissue could be at least one of bovine pericardium or porcine pericardium. The synthetic materials could be at least one of PTFE, polyester, silicone rubber, or polyurethanes. Moreover, the coverings can be in the form of continuous sheets, porous felts, or woven fabrics. One end of a length of the covering may be attached to stent 100 at or near attachment points 111, 121, while the other end of the covering may be attached to the stent at or near attachment points 115, 125. Attachment of the covering or other components to the stent may be accomplished by any known expedient, such as, for example, sutures, adhesives, ultrasonic or heat bonds, and the like.

In an alternative embodiment, an excess portion of the length of the covering may be left over once the covering has been attached to stent 100. For example, a portion spaced from one of the ends of the covering may be attached to or near the attachment points at that end, leaving a free end of the covering extending away from the stent. This free end can overlap with the opposite end of the covering to ensure that the covering fully encircles the surface of the stent when the stent is in a final expanded configuration. In some embodiments, a free end may be formed at both ends of the covering, and those free ends may overlap with one another in the final expanded configuration of the stent. In a further alternative, one type of covering material may be attached to body portion 110 (e.g., tissue material) and another type of covering material may be attached to cuff portion 120 (e.g., synthetic material). One or both of these coverings may be attached to the stent so as to provide a free end portion at one or both ends.

Since stent 100 is substantially planar in the initial configuration (whether expanded or compressed), the covering requires significantly less manipulation and coordination to be assembled in the desired manner to the stent. Moreover, any points on the stent or the covering at which attachment is to be made are readily accessible by hand or by attachment tools. This provides an improvement over prior methods of manufacturing stents in which the stents were in a non-planar (e.g., frustoconical) configuration, as manipulating the covering relative to the stent would be much more difficult, as would accessing all of the points of attachment, leading to an increased chance of error, poorer quality, as well as a lengthier manufacturing process.

Once stent 100 in the initial expanded configuration has been fully processed, the stent may be transformed to a final expanded configuration shown in FIG. 2. First attachment point 111 and second attachment point 115 may be joined to one another to form joined portion 130, while third attachment point 121 and fourth attachment point 125 may be joined to one another to form joined portion 140, as shown in FIG. 2. When the first attachment point is a hook 111a and the second attachment point is a receptacle 115a, as shown in FIG. 4, the hook may be inserted into an aperture in the receptacle to form joined portion 130a, securing ends 113a, 114a together. Ends 123, 124 may be joined to one another in a similar manner. Alternatively, as shown in FIG. 5, end 113b may be secured to end 114b by tying a knot 131b to form joined portion 130b. Knot 131b may be any known knot, and in particular, knots known in the art of suturing. A similar knot may be used to join ends 123, 124 to one another. In yet another alternative shown in FIG. 6, end 113c may be integrally secured to end 114c by a welding, soldering, brazing or other process known in the art to form joined portion 130c. Ends 123, 124 may be joined to one another using a similar process.

Once stent 100 is in the final expanded configuration, the stent may be shape set using a molten salt bath or other process known in the art for shape setting certain heat sensitive materials, such as Nitinol. This final expanded configuration is the configuration toward which stent 100 expands when deployed from a delivery device within the patient. The stent may then be collapsed to a compressed condition, such as that depicted in FIG. 3. Once collapsed, the stent 100 may be inserted into a catheter in preparation for implantation into a patient.

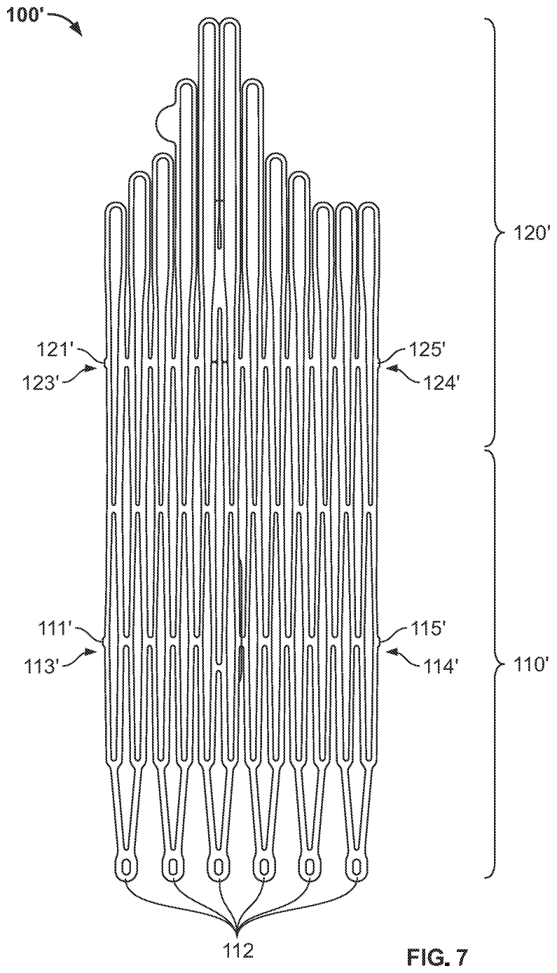

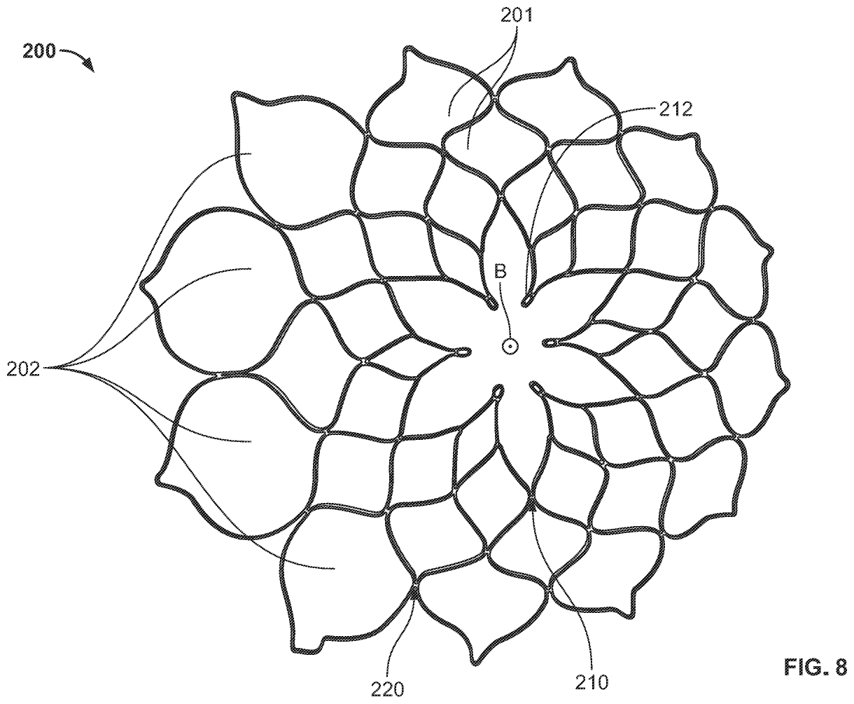

Stent 100 may have various sizes and/or shapes in the initial and final configurations. For example, stent 100 may have a substantially straight initial configuration, rather than the curved configuration shown in FIG. 1. FIG. 7 depicts stent 100′ in an initial compressed configuration that is straight, rather than curved. Stent 100′ is cut from the plate in a compressed condition while maintaining a substantially planar profile. While maintaining the substantially planar profile, stent 100′ may be transformed from the compressed condition to an expanded condition by pulling end 113′ away from end 114′, and end 123′ away from end 124′, or through the use of dies or other processes known in the art to change the shape of a stent. Stent 100′ may undergo this geometry change prior to being shape set and formed to a final expanded configuration. In its final expanded configuration, stent 100 or stent 100′ may have a frustoconical shape as described above and shown in FIG. 2, or another shape, such as cylindrical, or tubular with an oval or D-shaped transverse cross-section.

In a further example, the stent may be formed so as to not need attachment points 111, 115, 121, 125. FIG. 8 depicts stent 200 in an initial expanded configuration having an elliptical shape while maintaining a substantially planar profile. Stent 200 includes body portion 210 and cuff portion 220, both having a number of cells with openings 201 or 202. Openings 202 are larger than openings 201. In the embodiment shown in FIG. 8, there are four cells with larger openings 202 adjacent one another in the outermost row of cells, while the remainder of the cells in the stent have smaller openings 201. The larger openings 202 can be sized and shaped to account for movement for certain portions of the patient's anatomy once stent 200 is implanted, such as an anterior portion of the heart. In one example, larger openings 202 can allow for an atraumatic engagement of cuff portion 220 with the aorto-mitral curtain by providing a flexible structure for stent 200 to maintain its position once it has been implanted but will not interfere with the functions of the aortic valve during the movement of each cardiac cycle. The smaller openings 201 are stiffer due to their decreased size.

Stent 200 is cut from the plate so that body portion 210 and cuff portion 220 are both continuous without free ends that would require attachment to one another. Once stent 200 has been fully processed, the stent may be transformed to its final expanded configuration by manipulating certain portions of the stent in a direction transverse to the substantially planar profile of the stent in its initial expanded configuration. For example, engagement portions 212 may all be pulled or pushed in a direction perpendicular to the initial plane of the stent, converting the stent to a three-dimensional, generally tubular profile having a central axis B.

In a variant of the foregoing processes, other components configured to be assembled to stent 100, 200 such as an inner frame, can be manufactured in a manner similar to one of the methods described above for manufacturing the stent. For example, an inner frame configured to be assembled within stent 100, 200 may also be cut from a flat plate in an initial configuration with a planar profile and attachment points. In an alternative example, the inner frame can be cut from the plate without any attachment points so as to have a continuous elliptical shape, similar to stent 200. The inner frame may be assembled to stent 100 before the attachment points of the stent are joined together to form an implant in a final configuration or may be assembled to stent 200 in its initial, planar configuration after being cut from the plate. In another example, a valve assembly having a cuff or skirt and one or more valve leaflets may be assembled within stent 100 or stent 200 to form a prosthetic heart valve.

The manufacturing processes of this disclosure enables a stent to be manufactured with greater efficiency while minimizing inaccuracies and errors posed by cutting a stent from a round tube rather than from a substantially planar plate. For example, the planar profile of the stent in the initial configuration may enable the covering to be more easily and efficiently attached to the stent, particularly where the covering is to be positioned on the inner surface of the stent. Further, the planar profile of stent 100 in the initial configuration may allow a thinner covering material to be used. In such event, stent 100 may be compressible to a smaller diameter, allowing for smaller catheter sizes (e.g., from 36 Fr to 18 Fr) to be inserted into a patient without reducing the surface area covered by the stent when in the final expanded configuration. Moreover, cutting a stent from a plate so that the stent is already in an expanded configuration induces less strain in the stent than prior forming processes which require the stent structure to be expanded.

To summarize the foregoing, a stent includes a body portion having a first configuration and a second configuration, the body portion in the first configuration lying substantially in a plane and having a first end, a second end, a first attachment point at the first end, and a second attachment point at the second end, the body portion in the first configuration being curved about an axis substantially perpendicular to the plane; and/or
the stent may further include an engagement portion extending from the body portion substantially in a direction toward the axis; and/or
the stent may further include an additional component secured to the body portion; and/or
the stent may further include a cuff portion extending from the body portion, the cuff portion having a first configuration and a second configuration; and/or
the cuff portion in the first configuration may extend from the body portion in directions away from the axis; and/or
the cuff portion in the first configuration may be curved about the axis; and/or
the body portion in the first configuration may be at a first radial distance from the axis and the cuff portion in the first configuration may be at a second radial distance from the axis, the second radial distance being greater than the first radial distance; and/or
the cuff portion in the first configuration may lie substantially in the plane and may have a first end, a second end, a third attachment point at the first end and a fourth attachment point at the second end; and/or
the first attachment point may be connected to the second attachment point in the second configuration of the body portion to form a first joined portion; and/or
the body portion may be concentric about the axis in the second configuration; and/or
the first joined portion may be one of a hook received within a receptacle, a knot, or a welded connection; and/or
the stent may further include a covering engaged to at least one of an interior surface or an exterior surface of the body portion; and/or
the covering may be one of tissue or a synthetic material; and/or
a first end of the covering may be attached to the first attachment point, and at least one of a second end of the covering or a portion of the covering spaced from the second end of the covering may be attached to the second attachment point; and/or
a prosthetic heart valve may include a valve assembly and the foregoing stent.

According to another aspect of the disclosure, a method of forming a stent includes cutting a body portion of the stent from a substantially planar plate, the body portion in a first configuration being substantially planar and having a first end, a second end, a first attachment point at the first end, and a second attachment point at the second end, and connecting the first attachment point to the second attachment point to transform the body portion from the first configuration to a second configuration that is nonplanar; and/or
the method may further include securing an additional component to cover the body portion prior to connecting the first attachment point to the second attachment point; and/or
the method may further include shape setting the body portion after connecting the first attachment point to the second attachment point; and/or
the method may further include cutting a cuff portion of the stent from the plate, the cuff portion extending from the body portion; and/or
the cuff portion may be cut to have a first end, a second end, a third attachment point at the first end, and a fourth attachment point at the second end; and/or
the method may further include connecting the third attachment point to the fourth attachment point; and/or
the body portion may be cut so that the body portion in the first configuration is curved about an axis substantially perpendicular to a plane defined by the body portion; and/or
the body portion may be cut so as to include an engagement portion extending from the body portion substantially in a direction toward the axis; and/or connecting the first attachment point to the second attachment point may include transforming the body portion from the first configuration to the second configuration in which the body portion is concentric about the axis; and/or
the method may further include attaching a covering to the body portion prior to connecting the first attachment point to the second attachment point; and/or
attaching the covering may include attaching a first end of the covering to the first attachment point, and attaching at least one of a second end of the covering or a portion of the covering spaced from the second end to the second attachment point.

According to another aspect of the disclosure, a stent includes a body portion having a first configuration and a second configuration, the body portion in the first configuration lying substantially in a plane and having a continuous elliptical shape about an axis substantially perpendicular to the plane.

According to another aspect of the disclosure, a method of forming a stent includes cutting a body portion of the stent from a substantially planar plate, the body portion in a first configuration defining a plane and having an elliptical shape about an axis substantially perpendicular to the plane, and transforming the body portion from the first configuration to a second configuration by manipulating the body portion about the axis.

Although the subject matter herein has been described with reference to particular embodiments, it is to be understood that these embodiments are merely illustrative of the principles and applications of the subject matter described. It is therefore to be understood that numerous modifications may be made and that other arrangements may be devised without departing from the spirit and scope of the described subject matter as defined by the appended claims.

1. A stent, comprising:
a body portion having a first configuration and a second configuration, the body portion in the first configuration lying substantially in a plane and having a first end, a second end, a first attachment point at the first end, and a second attachment point at the second end, the body portion in the first configuration being curved about an axis substantially perpendicular to the plane;
a cuff portion extending from the body portion and lying substantially in the plane, the cuff portion having a first configuration and a second configuration, the cuff portion having a first end, a second end, a third attachment point at the first end and a fourth attachment point at the second end; and
a covering engaged to at least one of an interior surface or an exterior surface of the body portion, wherein a first end of the covering is attached to the first attachment point, and at least one of a second end of the covering or a portion of the covering spaced from the second end of the covering is attached to the second attachment.

2. The stent according to claim 1, further comprising an engagement portion extending from the body portion substantially in a direction toward the axis.

3. The stent according to claim 1, wherein the cuff portion in the first configuration extends from the body portion in directions away from the axis.

4. The stent according to claim 1, wherein the cuff portion in the first configuration is curved about the axis.

5. The stent according to claim 1, wherein the body portion in the first configuration is at a first radial distance from the axis and the cuff portion in the first configuration is at a second radial distance from the axis, the second radial distance being greater than the first radial distance.

6. The stent according to claim 1, wherein the first attachment point is connected to the second attachment point in the second configuration of the body portion to form a first joined portion.

7. The stent according to claim 1, wherein the body portion is concentric about the axis in the second configuration.

8. The stent according to claim 1, wherein the first joined portion comprises one of a hook received within a receptacle, a knot, or a welded connection.

9. The stent according to claim 1, further comprising a covering engaged to at least one of an interior surface or an exterior surface of the body portion.

10. The stent according to claim 9, wherein the covering comprises one of tissue or a synthetic material.

11. The stent according to claim 9, wherein a first end of the covering is attached to the first attachment point, and at least one of a second end of the covering or a portion of the covering spaced from the second end of the covering is attached to the second attachment point.

12. The stent according to claim 1, wherein the body portion in the first configuration has a continuous elliptical shape about an axis substantially perpendicular to the plane.

13. A prosthetic heart valve comprising:
a stent according to claim 1; and
a valve assembly secured to the stent.

14. A method of forming a stent, comprising:
cutting a body portion of the stent from a substantially planar plate, the body portion in a first configuration being substantially planar and having a first end, a second end, a first attachment point at the first end, and a second attachment point at the second end; and
connecting the first attachment point to the second attachment point to transform the body portion from the first configuration to a second configuration that is nonplanar.

15. The method according to claim 14, further comprising securing an additional component to cover the body portion prior to connecting the first attachment point to the second attachment point.

16. The method according to claim 14, further comprising shape setting the body portion after connecting the first attachment point to the second attachment point.

17. The method according to claim 14, further comprising cutting a cuff portion of the stent from the plate, the cuff portion extending from the body portion, the cuff portion having a first end, a second end, a third attachment point at the first end, and a fourth attachment point at the second end.

18. The method according to claim 17, further comprising connecting the third attachment point to the fourth attachment point.

19. The method according to claim 14, wherein the body portion is cut so that the body portion in the first configuration is curved about an axis substantially perpendicular to a plane defined by the body portion.

20. The method according to claim 14, wherein the body portion is cut so as to include an engagement portion extending from the body portion substantially in a direction toward the axis.

21. The method according to claim 14, wherein connecting the first attachment point to the second attachment point comprises transforming the body portion from the first configuration to the second configuration in which the body portion is concentric about the axis.

22. The method according to claim 14, further comprising attaching a covering to the body portion prior to connecting the first attachment point to the second attachment point.

23. The method according to claim 22, wherein attaching the covering comprises attaching a first end of the covering to the first attachment point, and attaching at least one of a second end of the covering or a portion of the covering spaced from the second end to the second attachment point.

24. The method according to claim 14, wherein the body portion has an elliptical shape about an axis substantially perpendicular to the plane, and
wherein the method further comprises transforming the body portion from the first configuration to a second configuration by manipulating the body portion about the axis.
